(19) United States
(12) Patent Application Publication
(10) Pub. No.: US 2024/0122541 A1
(43) Pub. Date: Apr. 18, 2024

(54) MULTI-MODULE WEARABLE DEVICE

(71) Applicant: ROCKLEY PHOTONICS LIMITED, Altrincham (GB)

(72) Inventors: Kate LeeAnn BECHTEL, Pleasant Hill, CA (US); Chia-Te CHOU, Brea, CA (US); Cody DUNN, Costa Mesa, CA (US); Armando MARTINEZ, Sherman Oaks, CA (US); David McCANN, Pasadena, CA (US); James McMILLAN, Santa Monica, CA (US); David Arlo NELSON, Fort Collins, CO (US); Andrew George RICKMAN, Marlborough (GB); Justin BECHSTEIN, Philadelphia, PA (US); Matt SELNICK, Philadelphia, PA (US); John TYRRELL, Philadelphia, PA (US); Jason ZERWECK, Philadelphia, PA (US)

(21) Appl. No.: 18/485,301

(22) Filed: Oct. 11, 2023

Related U.S. Application Data
(60) Provisional application No. 63/379,282, filed on Oct. 12, 2022.

(51) Int. Cl. A61B 5/00

(52) U.S. Cl. CPC A61B 5/681; A61B 2560/0443; A61B 2562/166; A61B 2562/222; A61B 2562/227

(57) ABSTRACT

A multi-module wearable device. According to an embodiment of the present disclosure, there is provided a system, including: a first wearable instrument; a second wearable instrument including a biometric sensor; an electrical connection between the first wearable instrument and the second wearable instrument; and a strap, sized and dimensioned to be disposed about a wrist. The electrical connection may be capable of connecting the first wearable instrument to the second wearable instrument when the second wearable instrument is at a first position on the strap relative to the first wearable instrument, and of connecting the first wearable instrument to the second wearable instrument when the second wearable instrument is at a second position on the strap relative to the first wearable instrument.

### CROSS-REFERENCE TO RELATED APPLICATION(S)

The present application claims priority to and the benefit of U.S. Provisional Application No. 63/379,282, filed Oct. 12, 2022, entitled “TWO-MODULE WEARABLE DEVICE”, the entire content of which is incorporated herein by reference.

### FIELD

One or more aspects of embodiments according to the present disclosure relate to biometric monitoring, and more particularly to a multi-module wearable device.

### BACKGROUND

A biometric monitoring system may be worn on the wrist of a subject, and may perform various biometric measurements on the dorsal side of the wrist. Some biometric measurements, e.g., ones which are based on measurements of arterial blood, may be performed on the volar side of the wrist.

It is with respect to this general technical environment that aspects of the present disclosure are related.

### SUMMARY

According to an embodiment of the present disclosure, there is provided a system, including: a first wearable instrument; a second wearable instrument including a biometric sensor; an electrical connection between the first wearable instrument and the second wearable instrument; and a strap, sized and dimensioned to be disposed about a wrist, the electrical connection being capable of: connecting the first wearable instrument to the second wearable instrument when the second wearable instrument is at a first position on the strap relative to the first wearable instrument; and connecting the first wearable instrument to the second wearable instrument when the second wearable instrument is at a second position on the strap relative to the first wearable instrument.

In some embodiments, the electrical connection includes a cable.

In some embodiments, the system is configured, when a separation between the first wearable instrument and the second wearable instrument is less than a length of the cable, to accommodate a surplus section of cable in a loop in an enclosure of the first wearable instrument.

In some embodiments, the system further includes a slider, in the enclosure of the first wearable instrument, for adjusting the size of the loop.

In some embodiments, the system is configured, when a separation between the first wearable instrument and the second wearable instrument is less than a length of the cable, to accommodate a surplus section of cable in a loop outside of an enclosure of the first wearable instrument.

In some embodiments, the system further includes a slider for adjusting the size of the loop.

In some embodiments, the slider is configured to be secured to the strap.

In some embodiments, the system is configured, when a separation between the first wearable instrument and the second wearable instrument is less than a length of the cable, to accommodate a surplus section of cable on a roller.

In some embodiments, the roller is in an enclosure of the first wearable instrument.

In some embodiments, the roller is configured to rotate about an axis parallel to a dorsal plane of the wrist.

In some embodiments, the roller is configured to rotate about an axis perpendicular to a dorsal plane of the wrist.

In some embodiments, the cable includes a flexible printed circuit board.

In some embodiments, a portion of the cable is covered by an overmold.

In some embodiments, the system is configured, when a separation between the first wearable instrument and the second wearable instrument is less than a length of the cable, to accommodate a surplus section of cable in a loop or on a roller.

In some embodiments, the surplus section is not covered by the overmold.

In some embodiments, the second wearable instrument is configured, when a separation between the first wearable instrument and the second wearable instrument is less than a length of the cable, to connect to the cable at a point on the cable, the point on the cable being less distant from the first wearable instrument than the length of the cable.

In some embodiments, the second wearable instrument is configured to make a connection with one or more discrete contacts of a plurality of discrete contacts extending along a portion of the cable.

In some embodiments, the second wearable instrument is configured to make a puncture connection with one or more conductors of the cable.

In some embodiments, the second wearable instrument is configured to make a capacitive connection with one or more conductors of the cable.

In some embodiments, the second wearable instrument is configured to make an inductive connection with one more or conductors of the cable.

### BRIEF DESCRIPTION OF THE DRAWINGS

These and other features and advantages of the present disclosure will be appreciated and understood with reference to the specification, claims, and appended drawings wherein:

FIG. 1A is a cross-sectional view of a system for monitoring biometrics, according to an embodiment of the present disclosure;

FIG. 1B is a cutaway perspective view of a portion of a system for monitoring biometrics, according to an embodiment of the present disclosure;

FIG. 1C is a cutaway perspective view of a portion of a system for monitoring biometrics, according to an embodiment of the present disclosure;

FIG. 1D is a perspective view of a system for monitoring biometrics, according to an embodiment of the present disclosure;

FIG. 1E is a cross-sectional view of a system for monitoring biometrics, according to an embodiment of the present disclosure;

FIG. 1F is a partially transparent plan view of a portion of a system for monitoring biometrics, according to an embodiment of the present disclosure;

FIG. 1G is a perspective view of a portion of a system for monitoring biometrics, according to an embodiment of the present disclosure;

FIG. 1H is a cross-sectional view of a portion of a system for monitoring biometrics, according to an embodiment of the present disclosure;

FIG. 1I is a partially transparent side view of a system for monitoring biometrics, according to an embodiment of the present disclosure;

FIG. 1J is a partially transparent perspective view of a portion of a system for monitoring biometrics, according to an embodiment of the present disclosure;

FIG. 2A is a cross-sectional view of a system for monitoring biometrics, according to an embodiment of the present disclosure;

FIG. 2B is a perspective view of a system for monitoring biometrics, according to an embodiment of the present disclosure;

FIG. 2C is a cross-sectional view of a system for monitoring biometrics, according to an embodiment of the present disclosure;

FIG. 2D is a perspective view of a system for monitoring biometrics, according to an embodiment of the present disclosure;

FIG. 2E is a side view of a system for monitoring biometrics, according to an embodiment of the present disclosure;

FIG. 2F is a side view of portion of a system for monitoring biometrics, according to an embodiment of the present disclosure;

FIG. 3A is a cross-sectional view of a system for monitoring biometrics, according to an embodiment of the present disclosure;

FIG. 3B is a cross-sectional view of a slider, according to an embodiment of the present disclosure;

FIG. 3C is a perspective view of a system for monitoring biometrics, according to an embodiment of the present disclosure;

FIG. 3D is a side view of a system for monitoring biometrics, according to an embodiment of the present disclosure;

FIG. 3E is a side view of a portion of a system for monitoring biometrics, according to an embodiment of the present disclosure;

FIG. 4A is a perspective view of a system for monitoring biometrics, according to an embodiment of the present disclosure;

FIG. 4B is a side view of a roller system for accommodating a surplus section of cable, according to an embodiment of the present disclosure;

FIG. 4C is a side view of a roller system for accommodating a surplus section of cable, according to an embodiment of the present disclosure;

FIG. 4D is a perspective view of a system for monitoring biometrics, according to an embodiment of the present disclosure;

FIG. 4E is a side view of a system for monitoring biometrics, according to an embodiment of the present disclosure;

FIG. 4F is a side view of a portion of a system for monitoring biometrics, according to an embodiment of the present disclosure;

FIG. 5 is a side cutaway view of a system for monitoring biometrics, according to an embodiment of the present disclosure;

FIG. 6A is a side cutaway view of a system for monitoring biometrics, according to an embodiment of the present disclosure;

FIG. 6B is a perspective view of a system for monitoring biometrics, according to an embodiment of the present disclosure;

FIG. 6C is a side view of a system for monitoring biometrics, according to an embodiment of the present disclosure;

FIG. 6D is a side view of a portion of a system for monitoring biometrics, according to an embodiment of the present disclosure;

FIG. 7 is a cross-sectional view of a system for monitoring biometrics, according to an embodiment of the present disclosure;

FIG. 8 is a cross-sectional view of a system for monitoring biometrics, according to an embodiment of the present disclosure;

FIG. 9 is a cross-sectional view of a system for monitoring biometrics, according to an embodiment of the present disclosure; and

FIG. 10 is a cross-sectional view of a system for monitoring biometrics, according to an embodiment of the present disclosure.

Each of the drawings is drawn to scale, for a respective embodiment.

### DETAILED DESCRIPTION

The detailed description set forth below in connection with the appended drawings is intended as a description of exemplary embodiments of a multi-module wearable device provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

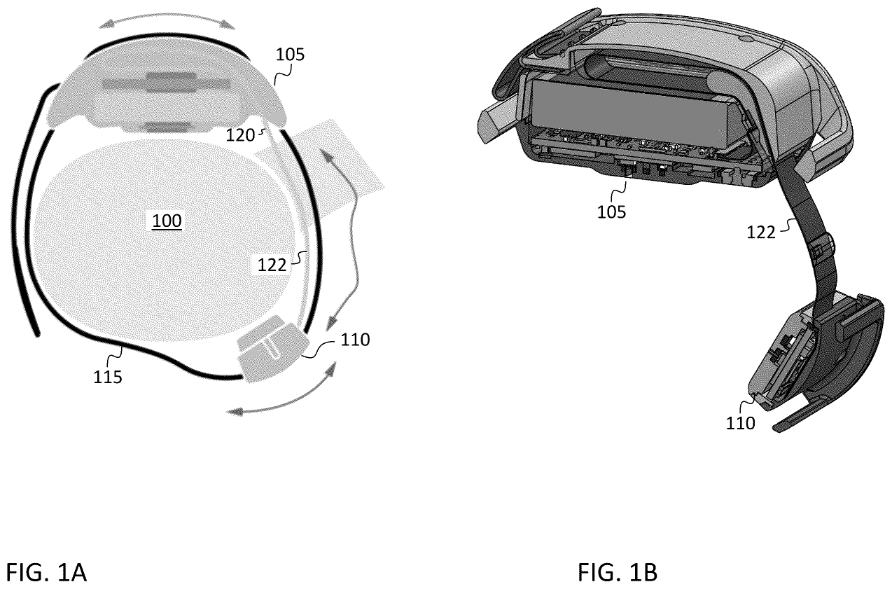

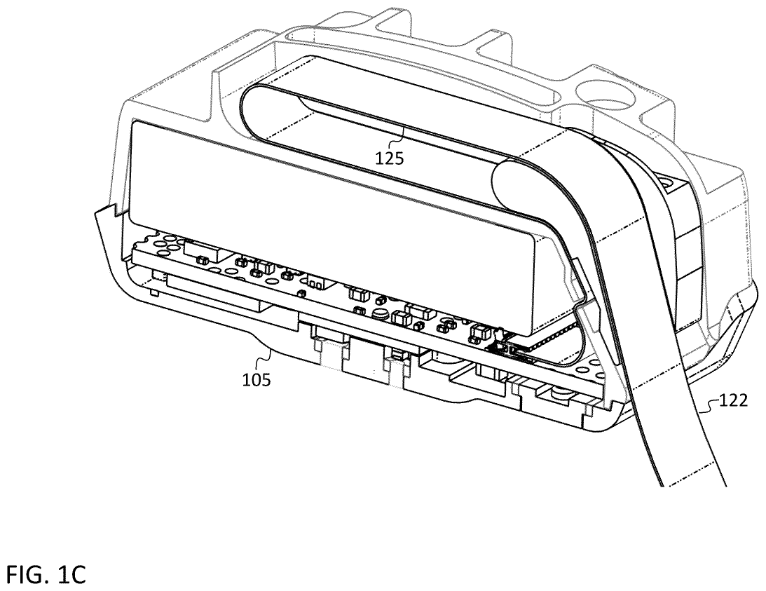

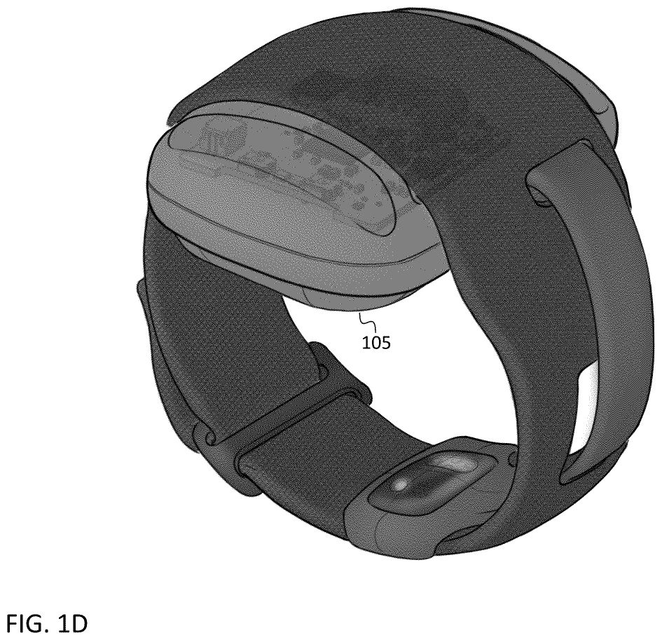

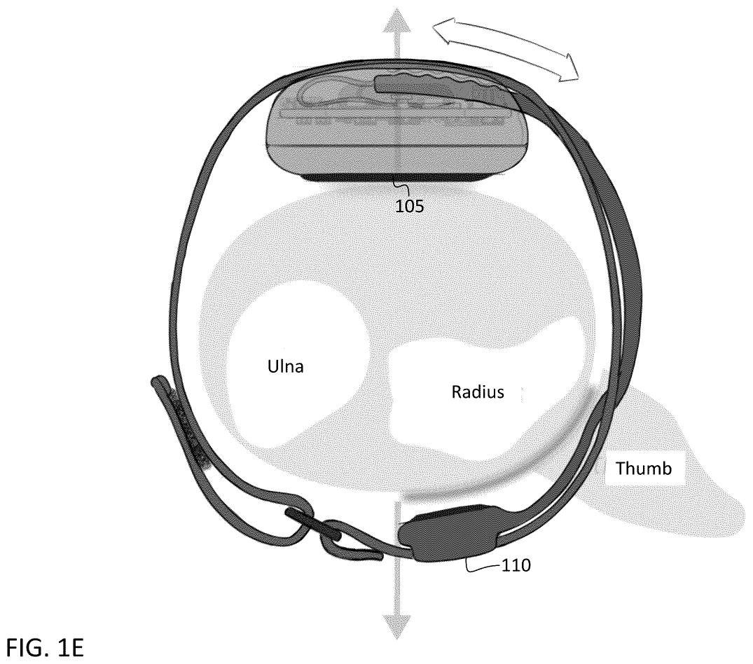

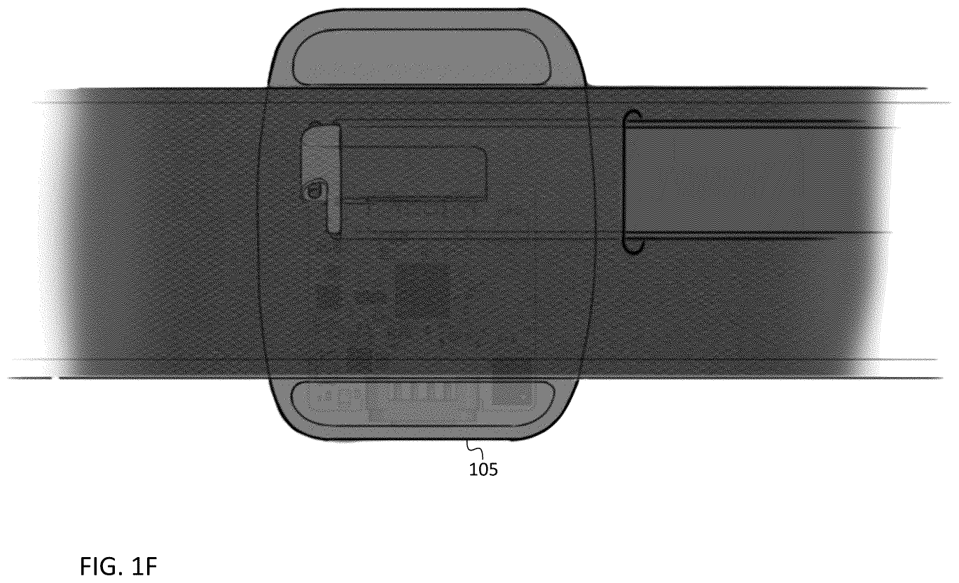

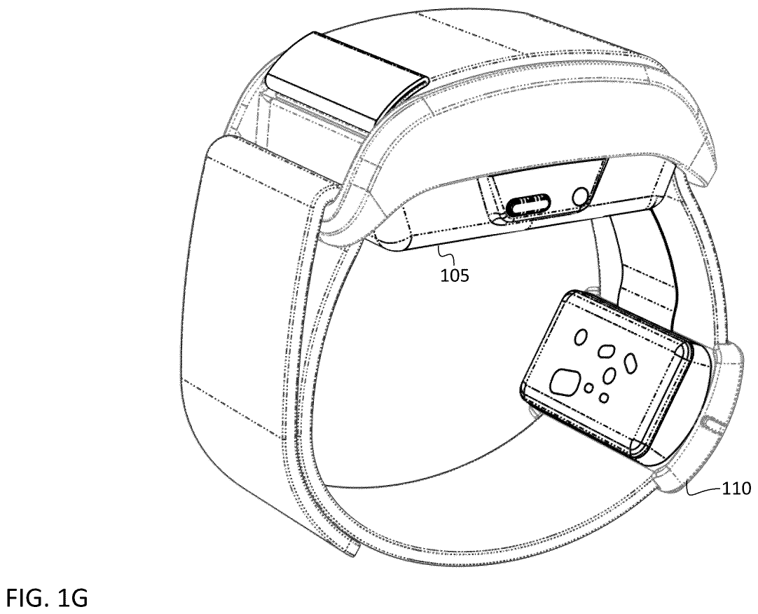

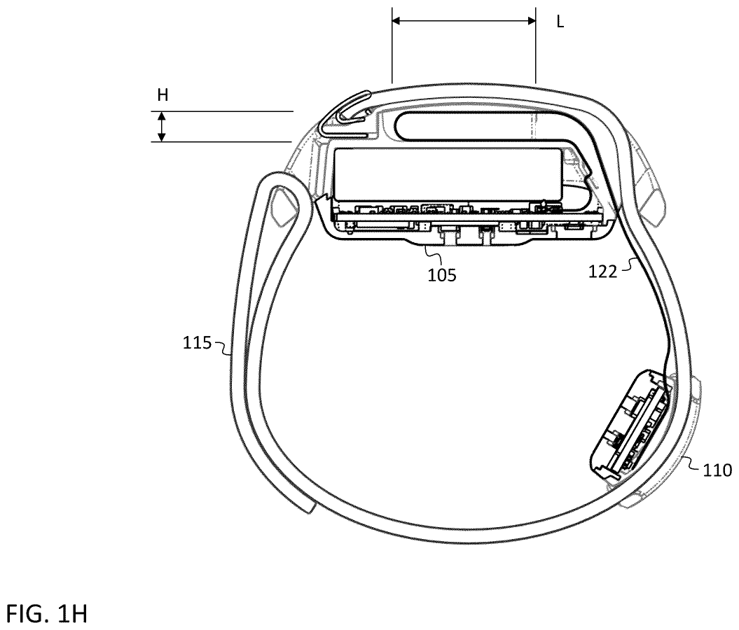

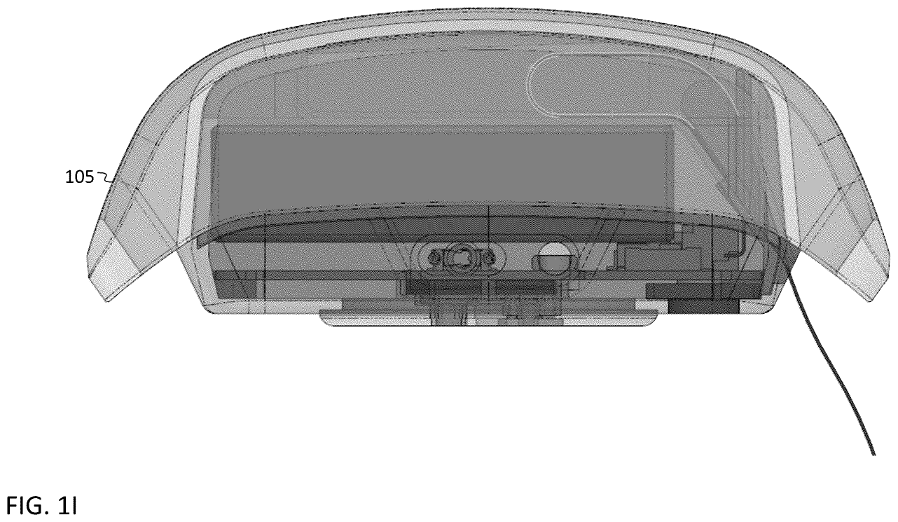

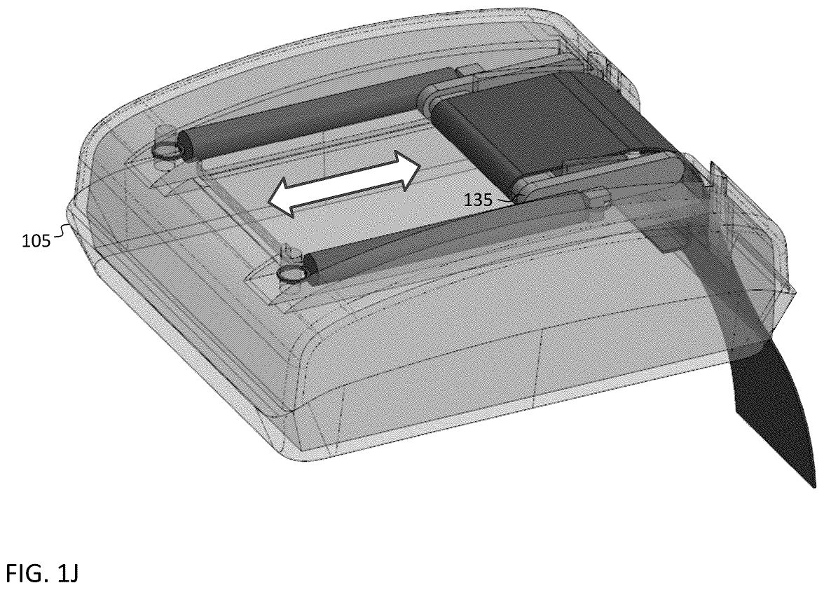

FIG. 1A shows a system for monitoring biometrics of a subject (e.g., of a patient). As used herein, “biometrics” are physiological parameters, examples of which are given below. The subject may wear the system of FIG. 1A on her or his wrist 100. The system may include, as illustrated, a first wearable instrument 105 and a second wearable instrument 110, both of which may be secured, in use, to the subject's wrist by a strap (e.g., a flexible strap) (or “band”) 115. The second wearable instrument 110 may be connected to the first wearable instrument 105 by an electrical connection 120 In some embodiments, the first wearable instrument 105 is larger than the second wearable instrument 110. The subject may be accustomed to wearing (and may prefer to wear) a wrist-worn device on the dorsal side (the top side) (e.g., in the dorsal central zone) of the wrist. As such, one of the wearable instruments may be worn on the dorsal side of the wrist, and, in embodiments in which one of the wearable instruments is larger than the other, the larger one of the wearable instruments (e.g., the first wearable instrument 105) may be worn on the dorsal side of the wrist. In such a configuration, various biometrics may be measured by the first wearable instrument 105. Some biometrics, however, are more readily measured or are only possible to be measured on the volar side (palm side) of the wrist where more direct access to arterial blood flow can be achieved.

As such, some embodiments include a first wearable instrument 105 and a second wearable instrument 110 (e.g., as illustrated in FIG. 1A), and the second wearable instrument 110 is independently repositionable with millimeter-scale resolution over a relatively large distance with respect to the first wearable instrument 105. For example, the system illustrated in FIG. 1A may be capable of securing the second wearable instrument 110 at any one of a plurality of positions (including, e.g., a first position, and a second position) along the strap 115, relative to the first wearable instrument 105. This adjustability may be made possible by adjustability of the position of the first wearable instrument 105 along the strap 115, or by adjustability of the position of the second wearable instrument 110 along the strap 115, or both. The adjustability may further be made possible by adjustability of the electrical connection 120, e.g., according to various embodiments described herein. As such, the electrical connection 120 may be capable of (i) connecting the first wearable instrument 105 to the second wearable instrument 110 when the second wearable instrument 110 is at a first position on the strap 115 relative to the first wearable instrument 105, and of (ii) connecting the first wearable instrument 105 to the second wearable instrument 110 when the second wearable instrument 110 is at a second position on the strap 115 relative to the first wearable instrument 105 (the separation, at the first position, between the first wearable instrument 105 and the second wearable instrument 110, differing from the separation at the second position, by, e.g., up to 38 mm (as discussed in further detail below)). Due to variations in human wrist circumference (which may vary, e.g., from 136 mm to 193 mm), the distance between the first wearable instrument 105 and the second wearable instrument 110, when the two wearable instruments are in respective optimal positions, may vary as much as 38 millimeters (mm) from a subject with a large wrist to a subject with a small wrist. The strap 115 may therefore be tightened or loosened (e.g., from 136 mm to 193 mm) to adjust to the size of the wrist of the subject, and the position of the second wearable instrument 110 relative to the first wearable instrument 105 on the strap 115 may be adjusted (e.g., independently adjusted, through a range of positions spanning 38 mm) so that the second wearable instrument 110 is at a position on the wrist at which acceptable signal quality may be obtained by one or more biometric sensors in the second wearable instrument 110. Such positions may be over a first relatively narrow region directly over the radial artery (or over a second relatively narrow region directly over the ulnar artery), and, as such, it may advantageous for the position of the second wearable instrument 110, relative to the position of the first wearable instrument 105, to be adjustable in relatively fine increments (e.g., increments of between 0.1 mm and 5.0 mm). Some embodiments include more than two wearable instruments, e.g., they may include three wearable instruments, of which one is configured to be worn on the dorsal side of the wrist, and two are configured to be worn on the volar side of the wrist.

The electrical connection 120 may include one or more (e.g., two or more) conductors, e.g., for transmitting power or signals between the first wearable instrument 105 and the second wearable instrument 110. The electrical connection 120 may include dedicated conductors or shared conductors (e.g., it may include a first power conductor, a first data conductor, and a shared ground conductor, the shared ground conductor being used both to form a power connection and a data connection). The electrical connection 120 may be employed for various functions. For example, the first wearable instrument 105 may include a battery supplying power both to (i) circuitry and one or more sensors in the first wearable instrument 105 and to (ii) circuitry and one or more sensors in the second wearable instrument 110. As another example, the first wearable instrument 105 may include a radio and an antenna for conducting wireless communications (e.g., Bluetooth™ or Wi-Fi™ communications) with stationary equipment (e.g., with a piece of medical equipment in a clinic or with a Wi-Fi router) or with a mobile device (e.g., a mobile telephone, laptop computer, or tablet computer), and the first wearable instrument 105 may relay signals (e.g., commands or data) to and from the second wearable instrument 110. The subject or a clinician may use such a mobile device for recording or relaying measurements obtained by the system (e.g., measurement data obtained by the first wearable instrument 105, or measurement data obtained by the second wearable instrument 110 and relayed by the radio of the first wearable instrument 105). In such embodiments, it may be unnecessary for the second wearable instrument 110 to include, for example, a battery, a radio, or an antenna, making it possible for the second wearable instrument 110 to be smaller than it otherwise would be. Other functions (e.g., a real-time clock, storage of subject-specific data, or encryption and decryption of data) may also be provided, for the second wearable instrument 110, by the first wearable instrument 105, to make further size reductions of the second wearable instrument 110 possible.

In some embodiments, as mentioned above, the first wearable instrument 105 is configured to be worn on the dorsal side of the wrist (and may be referred to as a “dorsal module”) and the second wearable instrument 110 is configured to be worn on the volar side of the wrist (e.g., over the radial artery or over the ulnar artery) (and may be referred to as a “volar module” 110). Some embodiments include an electrical connection 120 (which may be referred as a dorsal-volar link) between the dorsal module 105 and the volar module 110 that is capable of adjustment relative to the dorsal module 105. In order to ensure that the complete assembly does not have loose wires that would (i) make the sensing band unsightly, or prone to snagging, or (ii) interfere with sensor adjustment, the mechanism by which the dorsal-volar link is adjusted may be made to minimize the loose portions of the link either within the housing of the sensing modules or within the band. The terms “strap” and “band” are, as used herein, synonymous and they are used interchangeably. The strap 115 may be a single (e.g., fabric or elastomer) strip or it may include more than one strip of, e.g., fabric or elastomer. For example, it may include two pieces, connected to the first wearable instrument 105 in a manner similar to that of a two-piece watch band.

The present disclosure includes a series of embodiments describing how to electrically link two wrist worn sensing modules, e.g., a first wearable instrument 105 and a second wearable instrument 110, which may be referred to as the dorsal module 105 and the volar module 110 respectively, while at the same time providing the capability of adjusting the relative distance between the two sensing modules along the circumference of the wrist. Some embodiments (not including, e.g., ones employing puncture contacts) are re-adjustable and do not make use of adhesives. These embodiments are described here as respective numbered embodiments, for ease of description. Additional embodiments may readily be constructed by combining different features from the several numbered embodiments described herein. For example, the internal flex cable loop of Embodiment 1 may be combined with the partial overmold and protective sleeve of Embodiment 4.

A first embodiment, referred to as Embodiment 1, includes an internal flex cable loop, and is illustrated in FIGS. 1A-1J. In such an embodiment, the link between the dorsal module 105 and the volar module 110 is accomplished using a flexible printed circuit board (FPC) (or “flex cable”) 122. The flexible printed circuit board is connected to the rigid printed circuit boards (PCBs) of the dorsal module 105 and the volar module 110 by a suitable method (soldered or via a connector) or the rigid printed circuit and flexible printed circuit compose different parts of the same board (which may be referred to as a “rigid-flex” board). For example, a single printed circuit board may have a rigid portion (e.g., inside the dorsal module 105) including one or more rigid layers and one or more flexible layers. One or more of the flexible layers may extend beyond the edge of the rigid portion to form a flexible portion, connecting the dorsal module 105 and the volar module 110. The total length of the flexible printed circuit board 122 is made to accommodate the expected maximum required distance between the dorsal module 105 and the volar module 110. In order to allow for variation in the distance between the dorsal module 105 and the volar module 110, when not fully extended any unused portion of the flexible printed circuit board is contained within the dorsal module 105. The flexible printed circuit board is routed into a service loop configuration which is used to accommodate the surplus section of cable when the volar module 110 is less distant than it could be from the dorsal module 105. As such, the service loop may allow for adjustment of the relative distance of the volar module 110 from the dorsal module 105.

As illustrated in FIGS. 1B and 1C, the electrical connection 120 may be a cable, e.g., a flexible printed circuit board of fixed length, which includes an internal flex cable loop 125 accommodated within the first wearable instrument 105. Positional adjustment between the dorsal module 105 and the volar module 110 is achieved by insertion and extraction of flexible printed circuit board into and out of the dorsal module 105. The non-extracted length of flexible printed circuit board (which may be referred to as a surplus section of cable) is contained in the dorsal module service loop.

Steps for adjusting the system may include placing the loosened band over the wrist, tightening the band down (with loose tension on the sensor placement), shifting the sensor (adjusting the slider 135 as needed to take up the surplus section of cable), and adjusting the fabric band to secure the device in place. In such an embodiment, the use of a removable fabric band may enable easier cleaning, and a separate silicone band 124 for the flexible printed circuit board may be slim and discreet.

As illustrated in FIGS. 1D-1F, the cable 120 may include a flexible printed circuit board, a portion of which is covered with an overmold (e.g., an elastomer (e.g., silicone) overmold). This cable may pass through the fabric band. The portion of the flexible printed circuit board that is not covered with the overmold may form the service loop. The dorsal module 105 and volar module 110 may be aligned and the slack (the surplus section of cable) may move into the housing. The fabric band may be adjusted to create tension.

As illustrated in FIG. 1H, the loop height H may be, e.g., 4.5 mm, to prevent fatigue of the flexible printed circuit board and to reduce the adjustment force, and the cavity length may be 20 mm, to provide 40 mm of radial module adjustment. FIG. 1I shows an embodiment with a manual feed of the flexible printed circuit board into the cavity, and FIG. 1J shows an embodiment with a spring feed (e.g., with two 2.5 mm diameter springs configured to pull a slider 135 that pulls the loop into the cavity).

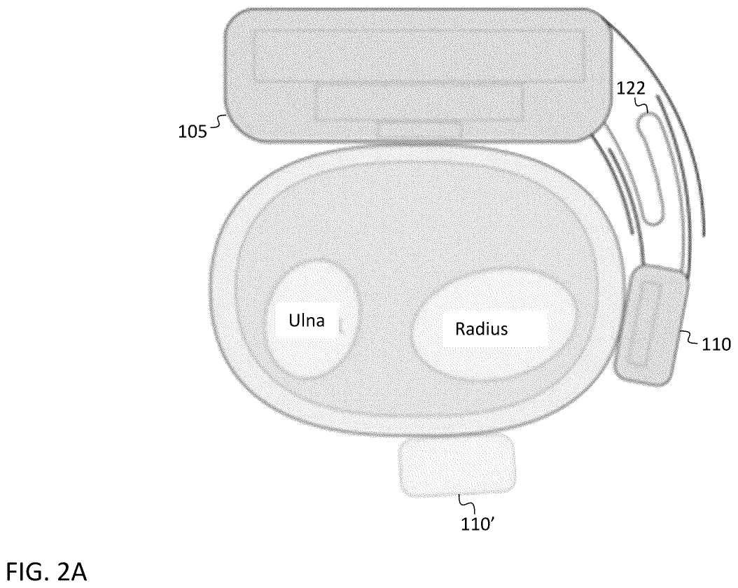

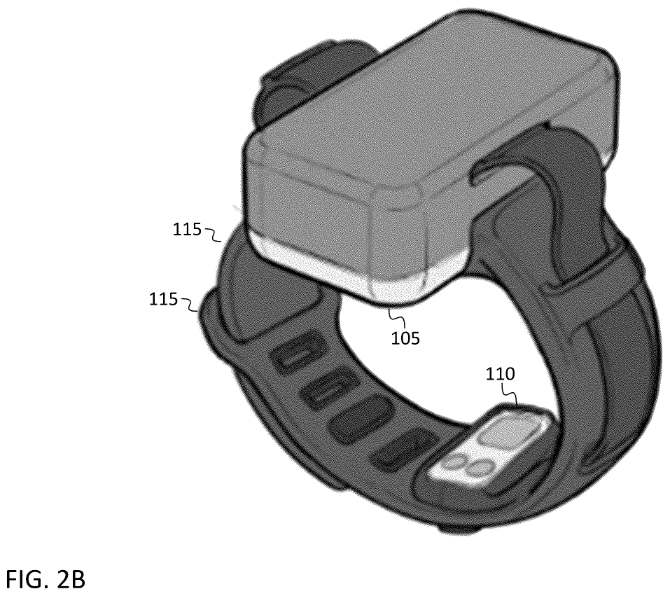

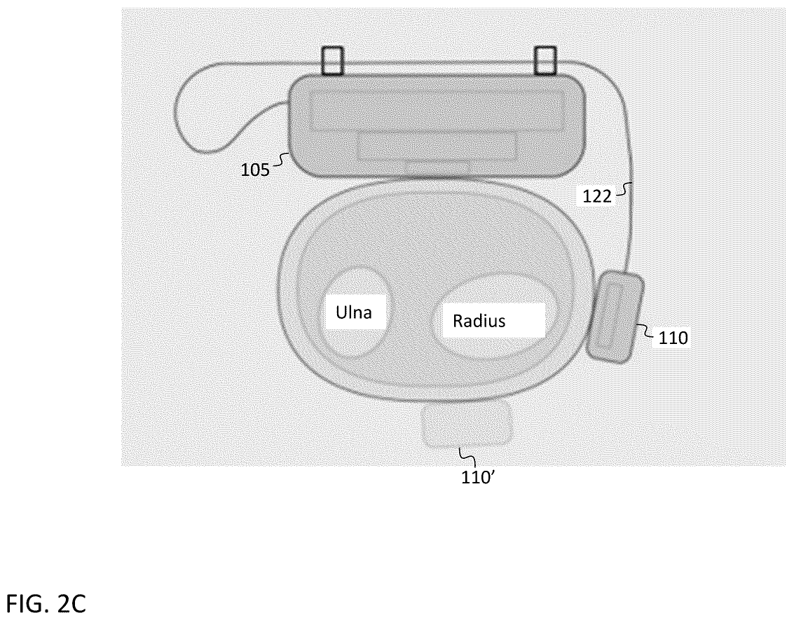

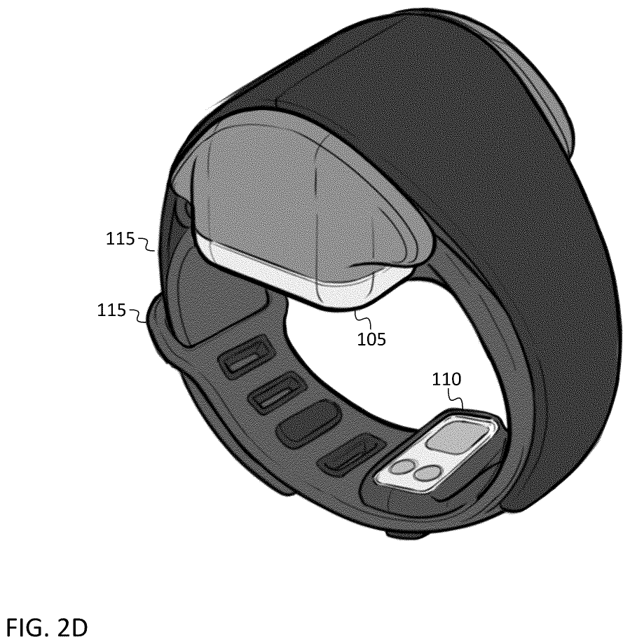

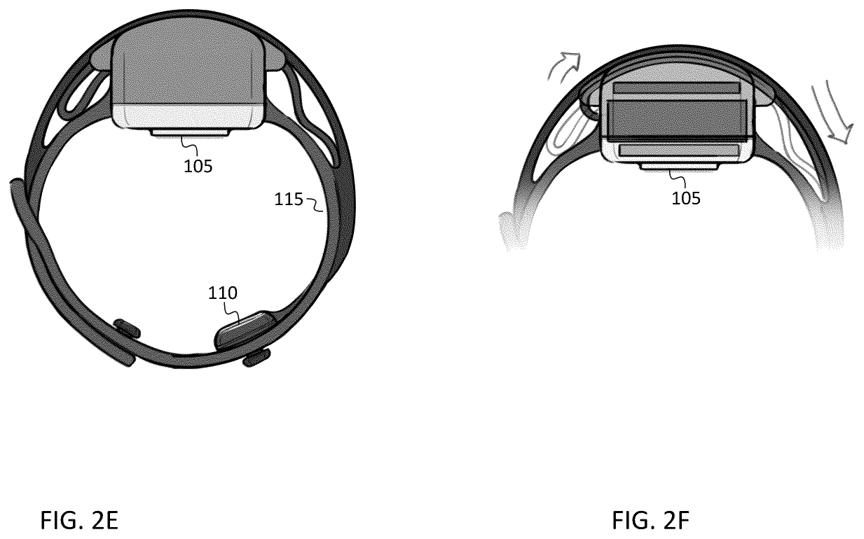

A second embodiment, referred to as Embodiment 2, includes an external flex cable loop. In this embodiment, as shown in FIGS. 2A-2C, the service loop may reside outside the dorsal module housing. This may decrease the dimensions of the dorsal module 105. The service loop may reside externally to the band in a separate housing. Alternatively, the service loop may reside in the band itself. FIGS. 2A and 2C show the volar module 110 both (i) in a first position close to the dorsal module 105 and (ii) (labeled 110′) in a second position more distant from the dorsal module 105.

In this embodiment, the electrical connection 120 between the dorsal module 105 and the volar module 110 is through flexible printed circuit board, the electrical contacts are fixed, and flexible printed circuit board is of a fixed length. Positional adjustment between the dorsal module 105 and the volar module 110 is achieved by coiling and unwrapping from a service loop, which holds the surplus section of cable. The service loop is external to the two modules.

As illustrated in FIGS. 2D-2F, adjustment steps may include loosely placing the band over the wrist, locating and securing the volar module 110 in place, so that the flexible printed circuit board adjusts to accommodate the location of the volar module 110, tightening and securing the tension band in place, checking the fit, and updating if necessary. In such an embodiment, the service loop may have minimal impact on the size of the dorsal module 105, and the service loop automatically adapts to the separation between the dorsal module 105 and the volar module 110. FIG. 2F shows the loop for accommodating the surplus section of cable for each of two positions of the volar module 110: a first position at or near the greatest possible distance from the dorsal module 105, and a second position, (shown in fainter lines in FIG. 2F) at a position that is nearer the dorsal module 105.

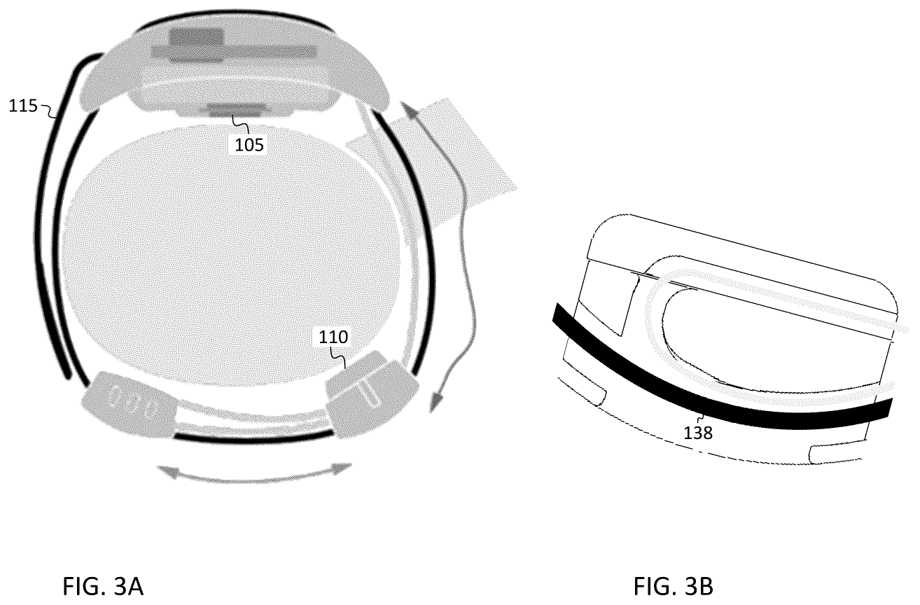

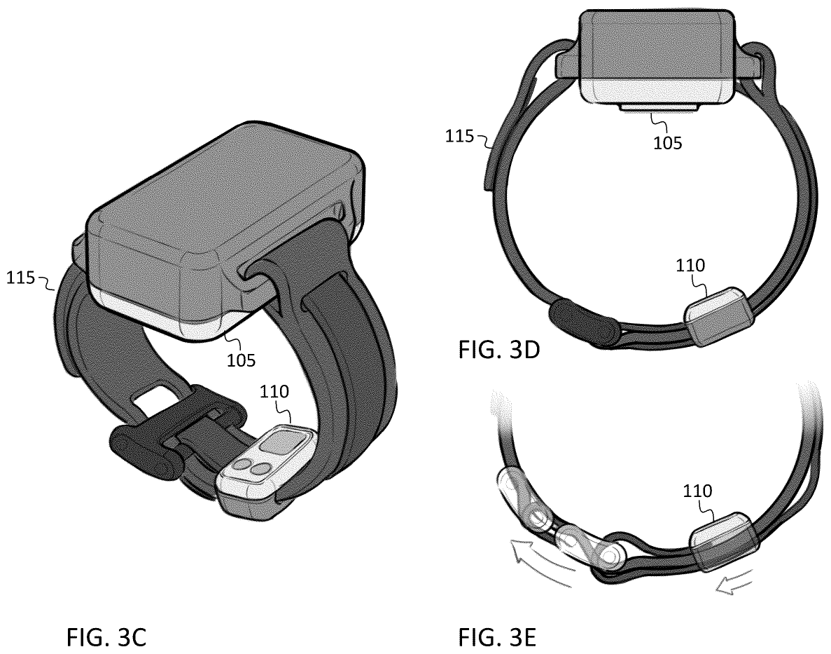

A third embodiment, referred to as Embodiment 3 and illustrated in FIGS. 3A-3E, utilizes a service loop external to the housing of the dorsal module 105 and employs a slider 135. The electrical connection 120 between dorsal module 105 and the volar module 110 includes a flexible printed circuit board. The slider 135 provides a mechanism by which the volar module 110 can be positioned with respect to the dorsal module 105 and the flexible printed circuit board can be maintained taut, following the shape of the band. The service loop between the volar module 110 and the slider 135 is responsible for accommodating the slack length of the flexible printed circuit board (the surplus section of cable) that may result from positional adjustment of the volar module 110 with respect to the dorsal module 105.

In this embodiment, the electrical contacts are fixed and the flexible printed circuit board is of a fixed length. The positional adjustment between the dorsal module 105 and the volar module 110 is achieved by coiling and unwrapping from the service loop. The service loop is external to the two modules. Steps for adjusting the system may include, as illustrated in FIGS. 3C-3E, loosely placing the band over the wrist, tightening and securing the tension band in place, sliding the volar module 110 along the band until located (the flexible printed circuit board may tent or pull during adjustment), and adjusting the slider 135 to take up any excess (the surplus section of cable). In such an embodiment, the service loop has minimal impact on dorsal module size, the slider 135 provides intuitive service loop management, and sliding features allow adjustment while the strap is tight. FIG. 3B shows a cap 138 that may be included in the slider 135 to guide the flexible printed circuit board out of the slider 135.

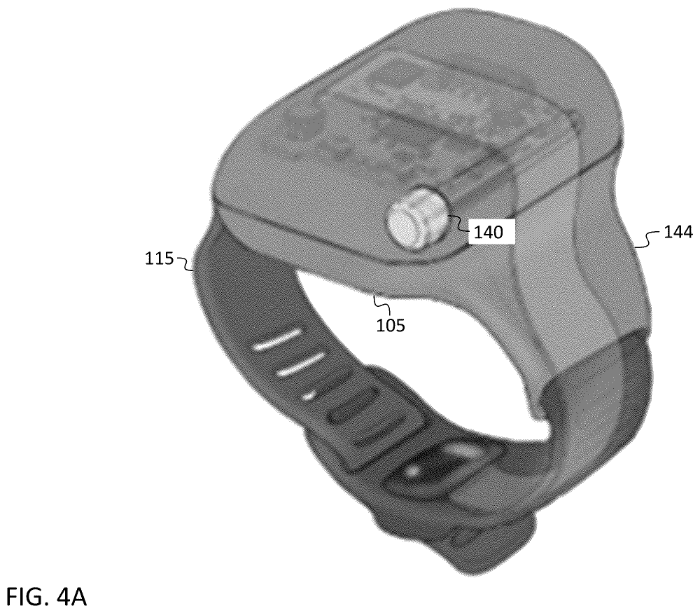

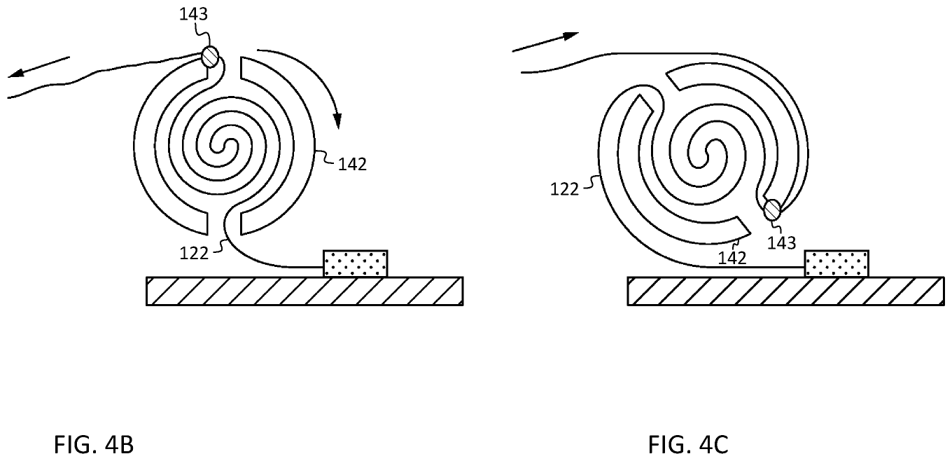

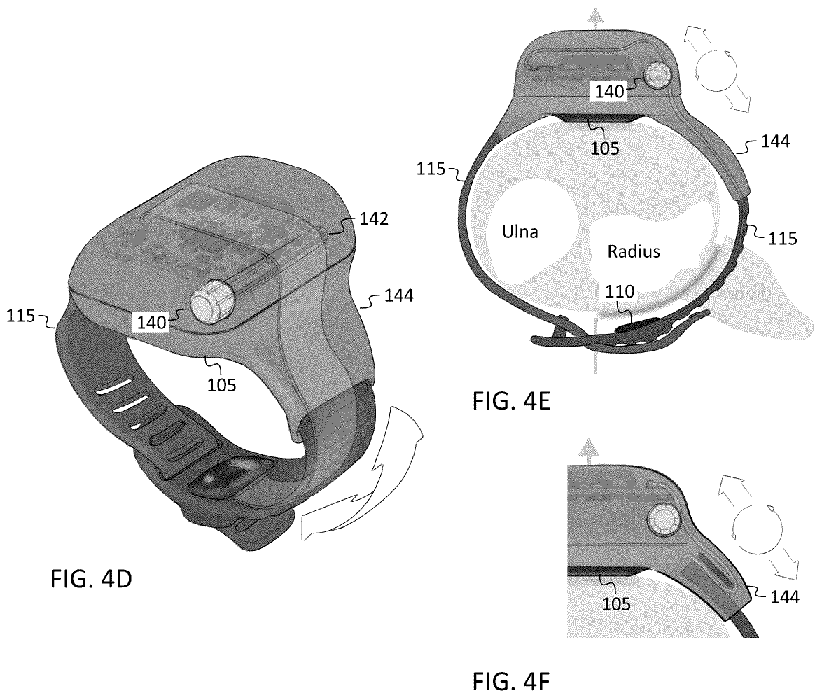

A fourth embodiment, referred to as Embodiment 4, includes a roller facilitated flex cable loop), as illustrated in FIGS. 4A-4F. This embodiments utilizes a roller to accommodate the slack length of the flexible printed circuit board (the surplus section of cable). The electrical connection 120 between dorsal module 105 and the volar module 110 includes a flexible printed circuit board. The flexible printed circuit board may be anchored to the roller 142 (at anchor points 143), and wound about the roller 142. The rotation of the roller may result in extension and retraction of the flexible printed circuit board from the roller. The roller can be manually actuated, like a watch crown, or be made to incorporate a spring mechanism which provides retraction tension. Some embodiments may employ a clutch mechanism on the roller so that the spring retraction tension is not continuously present but can be triggered by a small amount of retraction like that employed in roller blinds.

In Embodiment 4, the electrical contacts are fixed and the flexible printed circuit board is of a fixed length. The positional adjustment between the dorsal module 105 and the volar module 110 is achieved by rolling and unrolling flexible printed circuit board from a roller 142 contained in the dorsal module 105. The roller 142 may rotated about an axis substantially parallel to the dorsal plane and to the forearm of the user. In such an embodiment, the roller 142 may be manually adjusted via externally accessible knob 140 or handle, or the roller 142 may utilize a spring mechanism to facilitate retraction of the flexible printed circuit board. The system may include an internal and external coil.

As in Embodiment 2, a portion of the cable 120 may include a flexible printed circuit board with a silicone overmold. In Embodiment 4, a portion of the flexible printed circuit board may be left uncoated (i.e., free of the silicone overmold) so that it is able to be wound up on the roller 142 as needed. The housing of the dorsal module 105 may include a sleeve 144 that protects the uncoated portion of the flexible printed circuit board.

In this embodiment, the electrical contacts are fixed and the flexible printed circuit board is of a fixed length. Steps for adjusting the system may include placing the un-looped band onto the wrist, with the strap on the volar module side extended all the way out, looping the opposing side of band through, then tensioning it, shifting the volar module 110 (which may require taking up an additional surplus section of cable, if the volar module 110 is moved toward the dorsal module 105, or releasing some of the surplus section of cable if the volar module 110 is moved away from the dorsal module 105), adjusting the silicone band and securing the clasp. Such an embodiment may hide and protect the flexible printed circuit board from view and damage.

A fifth embodiment, referred to as Embodiment 5, includes a retractable cable loop, in an embodiment in which the link between the dorsal module 105 and the volar module 110 employs a multiconductor cable 123 with, e.g., a round cross section, and the axis of rotation of the roller 142 is rotated, relative to the roller 142 of Embodiment 4. In this embodiment the cable resides on a roller that rotates about an axis normal to the dorsal plane. The size of the roller can utilize the internal perimeter of the dorsal module 105 and the roller may be made hub-less so that the internal volume of the roller may be utilized while the cable resides on the outer side of the roller. In this manner the space of the internal volume of the roller may be used for components within the dorsal module 105.

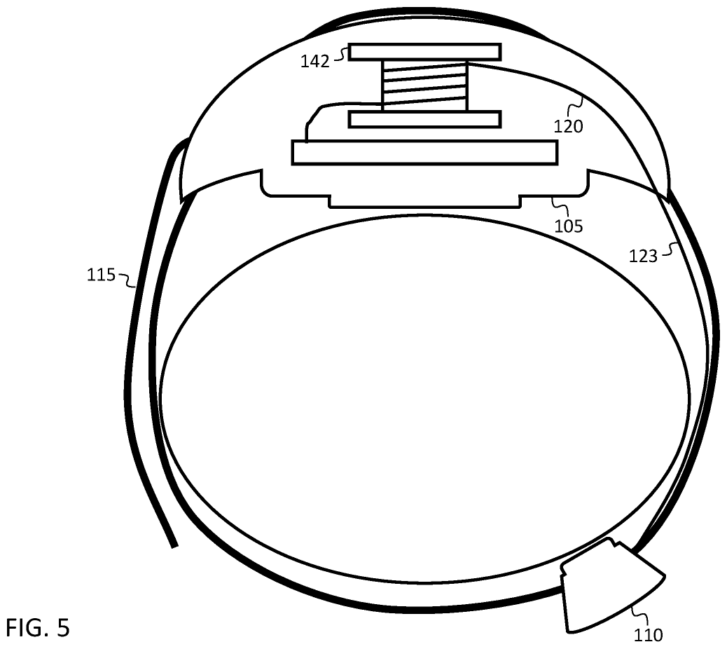

As illustrated in FIG. 5, the electrical connection 120 between the dorsal module 105 and the volar module 110 is through multiconductor cable. In Embodiment 5, the electrical contacts are fixed and cable is of a fixed length. Positional adjustment between the dorsal module 105 and the volar module 110 is achieved by rolling and unrolling cable from the roller 142, or “cable reel”, contained in the dorsal module 105. The cable reel may encircle the inside perimeter of the dorsal module 105 allowing functional components to reside within its internal space while the cable and reel assembly rotate around them. A coil inside the cable reel may be used to make a connection to the cable on the exterior of the spool.

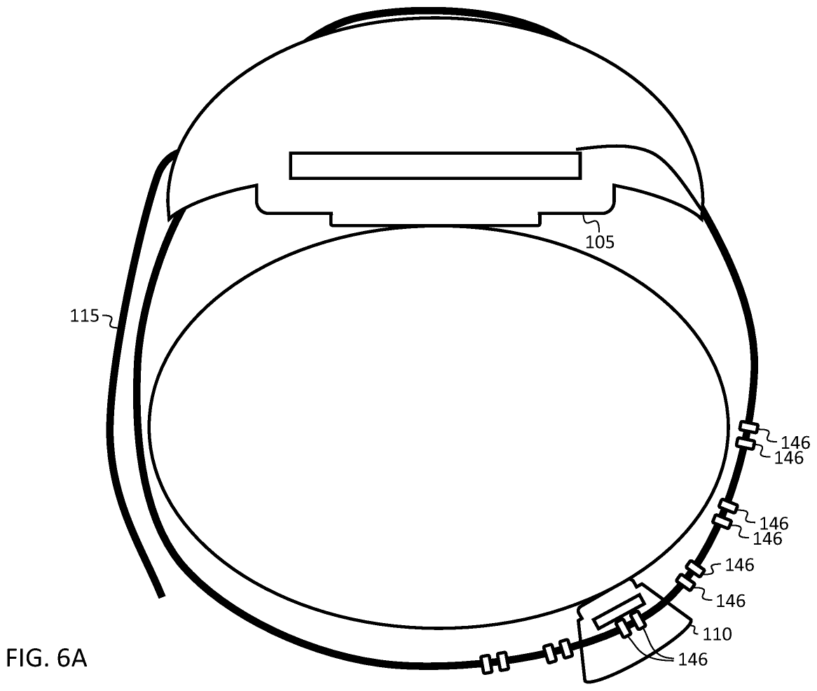

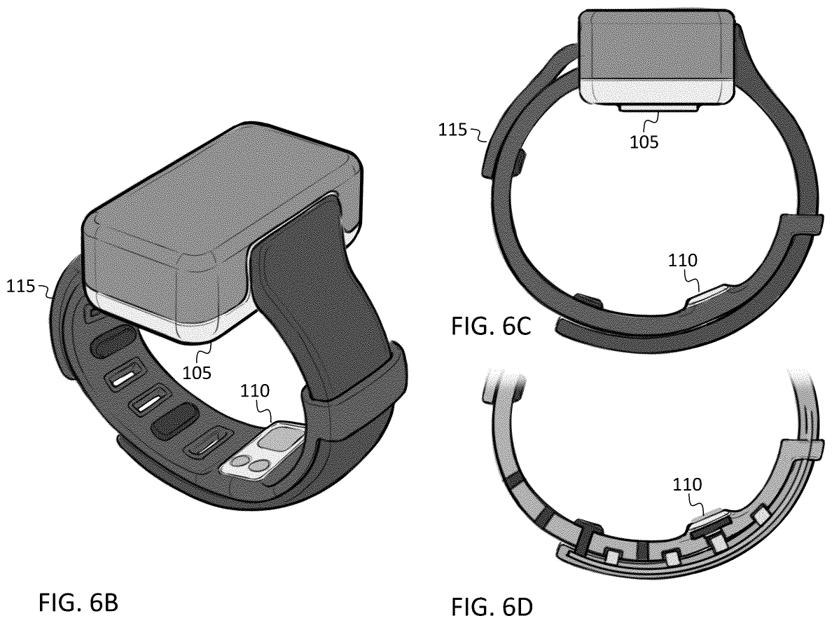

A sixth embodiment, referred to as Embodiment 6 and illustrated in FIGS. 6A-6D, includes discrete position contacts 146. Such an embodiment may utilize a flexible printed circuit board or flexible cable assembly that has a series of discrete connection points along its length to which the volar module 110 may attach. Each connection point represents a set of contacts 146 for each electrical conductor that makes up the electrical connection 120 between the dorsal and volar module 110. The electrical contacts may be mated by way of spring-loaded pins. This embodiment may not offer continuous positional adjustment but adjustment in discrete steps equal to the separation of the connection points. In such an embodiment, the electrical connection 120 between the dorsal module 105 and the volar module 110 may be through multiconductor cable or flexible printed circuit board. Electrical contacts are distributed along the length of the band at discrete positions. Positional adjustment between the dorsal module 105 and the volar module 110 may be achieved by connecting the volar module 110 to one of the discrete contact positions that are distributed along the length of the band.

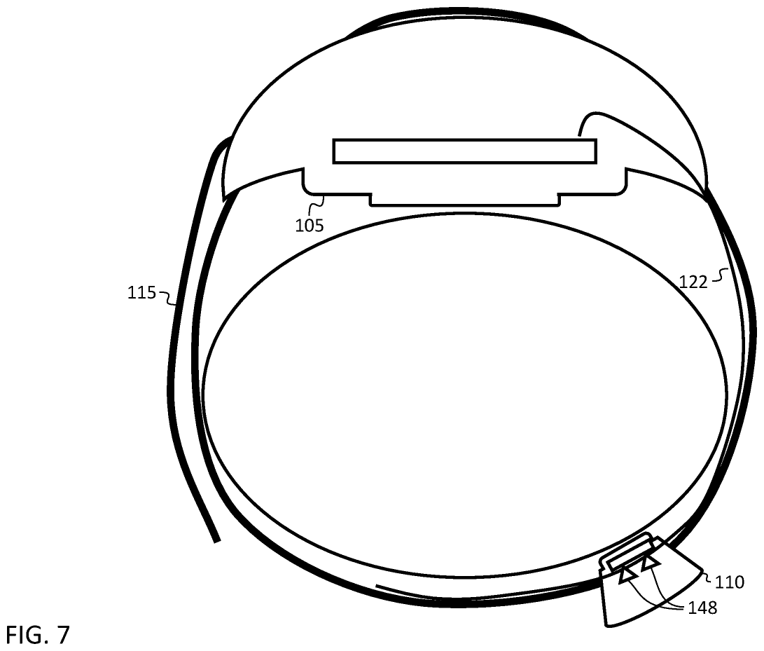

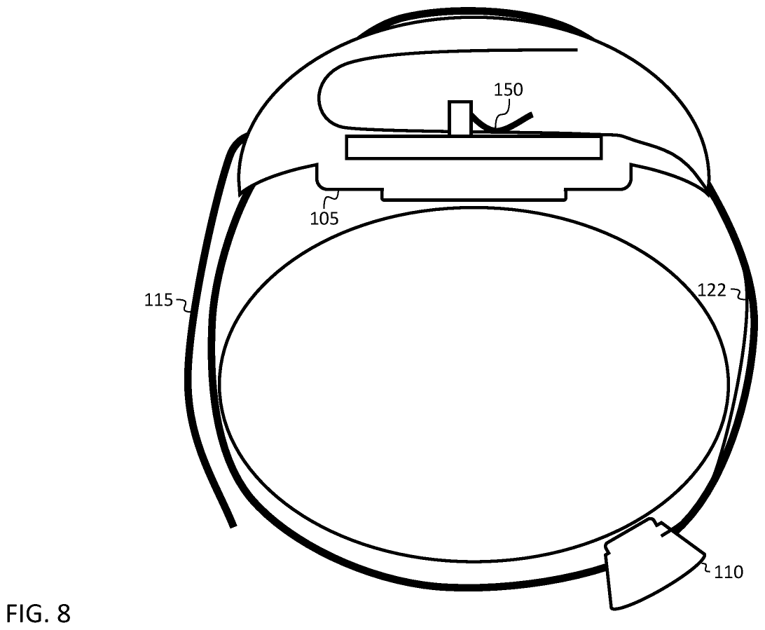

A seventh embodiment, referred to as Embodiment 7 and illustrated in FIG. 7, may employ a one-time-use series of puncture contacts 148 that pierce a flexible printed circuit board or other multiconductor cable assembly. Upon identification of the appropriate separation between the dorsal module 105 and the volar module 110, the volar module 110 may then be fixed in place by the puncture contacts 148. In this embodiment the link between the dorsal module 105 and the volar module 110 may be a consumable that may be replaced for each user of the device. The electrical connection 120 between the dorsal module 105 and the volar module 110 may be through a flat multiconductor cable or flexible printed circuit board. Individual wires or traces may be distributed continuously along the length of the band. Positional adjustment between the dorsal module 105 and the volar module 110 may be achieved by the puncture contacts 148 (e.g., single use puncture contacts) that pierce the cable or flexible printed circuit board and make electrical contact with the conductor. Each individual conductor may have its own puncture contact.

In an eighth embodiment, referred to as Embodiment 8 and illustrated in FIG. 8, electrical contact to the flexible printed circuit board is made using spring loaded sliding contacts 150 that allow the flexible printed circuit board to be extracted from or retracted into the dorsal module 105. The flexible printed circuit board may have exposed pads that the sliding contacts 150 mate with. As the flexible printed circuit board is extracted from or retracted into the dorsal module 105 during adjustment of the position of the volar module 110, the flexible printed circuit board may be constrained so that its exposed contacts slide underneath the spring loaded sliding contacts 150. In such an embodiment, the electrical connection between the dorsal module 105 and the volar module 110 is through a flexible printed circuit board with exposed conductors. The flexible printed circuit board is attached to the volar module 110 by, e.g., soldering, or a connector. The dorsal module 105 makes contact with the flexible printed circuit board via the exposed contacts using a series of sliding contacts. Positional adjustment between the dorsal module 105 and the volar module 110 may achieved by insertion of the flexible printed circuit board into, or extraction of the flexible printed circuit board from, the dorsal module 105, within which the non-extended flexible printed circuit board (the surplus section of cable) may be contained in service loop (as illustrated) or on a roller.

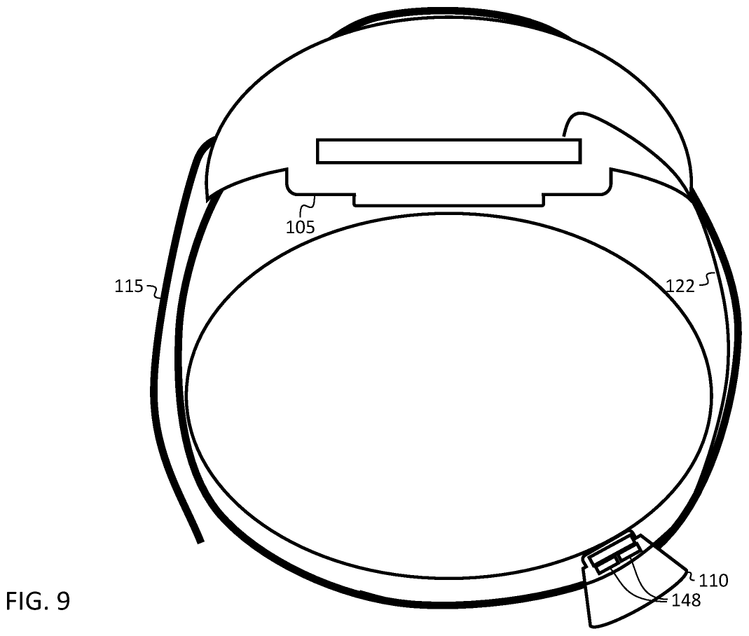

A ninth embodiment, referred to as Embodiment 9 and illustrated in FIG. 9, may use non-contact (e.g., capacitive) coupling between one or more conductors of the electrical connection 120 and one or more conductors of the volar module 110. For example, the volar module 110 may contain capacitive contacts 152, which may, in operation, be near, and capacitively coupled to, respective corresponding conductors of the flexible printed circuit board. The flexible printed circuit board from the dorsal module 105 may have mating capacitive plates distributed along its length for each required signal as well as near field transmitting coils for power. The flexible printed circuit board is attached to the dorsal module 105 by, e.g., soldering, or a connector.

The non-contact capacitive contacts 152 may interface with traces in the flexible printed circuit board. The volar module 110 may be powered by its own internal power supply (e.g., a battery in the volar module 110), by a compliant cable, or by near-field (inductive) power transfer, from one or more current loops in the flexible printed circuit board to one or more receiving coils in the volar module 110.

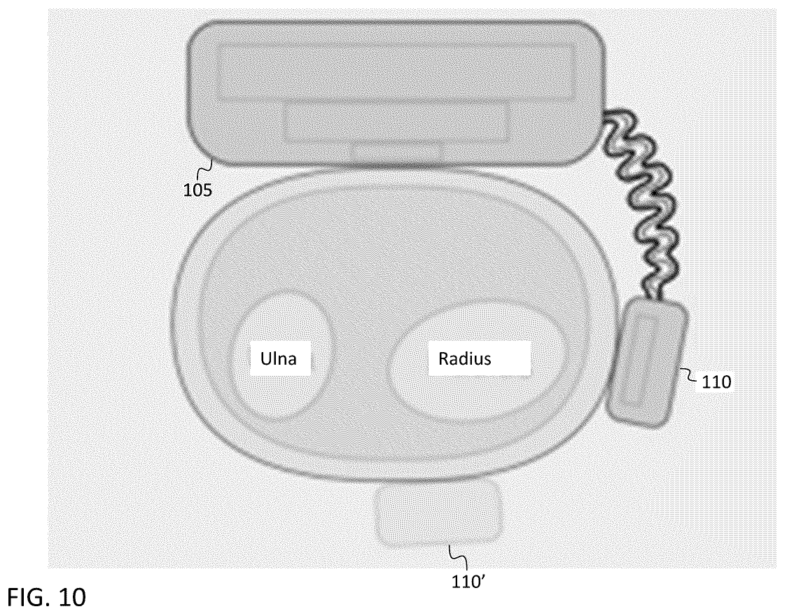

A tenth embodiment, referred to as Embodiment 10 and illustrated in FIG. 10, may employ a compliant cable between the dorsal module 105 and the volar module 110. The compliant cable may be a flexible printed circuit board or, e.g., a substantially round multiconductor cable capable of elongation and returning to its original shape. Example of such cables include but are not limited to serpentine flexible printed circuit boards and coiled multiconductor cables. The lengthwise compliance of the flexible printed circuit board may be achieved by out of plane (normal to the plane of the printed circuit) corrugations of the flexible printed circuit board or a flat (non-corrugated) serpentine shaped printed circuit board. The cable may be permanently attached to both the dorsal module 105 and the volar module 110 or may make use of an ingress protection (IP) rated connector to prevent ingress of foreign substances into the module.

In some embodiments, the system includes more than one volar module 110. For example, both the first wearable instrument 105 and the second wearable instrument 110 may be configured to be worn on the volar side of the wrist, or the system may include more than two wearable instruments, of which two or more are configured to be worn on the volar side of the wrist. In a system with two wearable instruments, both of which are configured to be worn on the volar side of the wrist, an electrical connection (e.g., an adjustable connection according to one of the embodiments disclosed herein, or according to an embodiment combining features of several embodiments disclosed herein) may connect the two wearable instruments. In a system with more than two wearable instruments, one or more pairs of the wearable instruments may be connected together with an electrical connection according to one of the embodiments disclosed herein, or according to an embodiment combining features of several embodiments disclosed herein.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a multi-module wearable device have been specifically described and illustrated herein, many modifications and variations will be apparent to those skilled in the art. Accordingly, it is to be understood that a multi-module wearable device constructed according to principles of this disclosure may be embodied other than as specifically described herein. The invention is also defined in the following claims, and equivalents thereof.

What is claimed is:

1. A system, comprising:
a first wearable instrument;
a second wearable instrument comprising a biometric sensor;
an electrical connection between the first wearable instrument and the second wearable instrument; and
a strap, sized and dimensioned to be disposed about a wrist,
the electrical connection being capable of:
connecting the first wearable instrument to the second wearable instrument when the second wearable instrument is at a first position on the strap relative to the first wearable instrument; and
connecting the first wearable instrument to the second wearable instrument when the second wearable instrument is at a second position on the strap relative to the first wearable instrument.

2. The system of claim 1, wherein the electrical connection comprises a cable.

3. The system of claim 2, wherein the system is configured, when a separation between the first wearable instrument and the second wearable instrument is less than a length of the cable, to accommodate a surplus section of cable in a loop in an enclosure of the first wearable instrument.

4. The system of claim 3, further comprising a slider, in the enclosure of the first wearable instrument, for adjusting the size of the loop.

5. The system of claim 2, wherein the system is configured, when a separation between the first wearable instrument and the second wearable instrument is less than a length of the cable, to accommodate a surplus section of cable in a loop outside of an enclosure of the first wearable instrument.

6. The system of claim 5, further comprising a slider for adjusting the size of the loop.

7. The system of claim 6, wherein the slider is configured to be secured to the strap.

8. The system of claim 2, wherein the system is configured, when a separation between the first wearable instrument and the second wearable instrument is less than a length of the cable, to accommodate a surplus section of cable on a roller.

9. The system of claim 8, wherein the roller is in an enclosure of the first wearable instrument.

10. The system of claim 9, wherein the roller is configured to rotate about an axis parallel to a dorsal plane of the wrist.

11. The system of claim 9, wherein the roller is configured to rotate about an axis perpendicular to a dorsal plane of the wrist.

12. The system of claim 2, wherein the cable comprises a flexible printed circuit board.

13. The system of claim 12, wherein a portion of the cable is covered by an overmold.

14. The system of claim 13, wherein the system is configured, when a separation between the first wearable instrument and the second wearable instrument is less than a length of the cable, to accommodate a surplus section of cable in a loop or on a roller.

15. The system of claim 14, wherein the surplus section is not covered by the overmold.

16. The system of claim 2, wherein the second wearable instrument is configured, when a separation between the first wearable instrument and the second wearable instrument is less than a length of the cable, to connect to the cable at a point on the cable, the point on the cable being less distant from the first wearable instrument than the length of the cable.

17. The system of claim 16, wherein the second wearable instrument is configured to make a connection with one or more discrete contacts of a plurality of discrete contacts extending along a portion of the cable.

18. The system of claim 16, wherein the second wearable instrument is configured to make a puncture connection with one or more conductors of the cable.

19. The system of claim 16, wherein the second wearable instrument is configured to make a capacitive connection with one or more conductors of the cable.

20. The system of claim 16, wherein the second wearable instrument is configured to make an inductive connection with one more or conductors of the cable.
